(19) United States
(12) Patent Application Publication
(10) Pub. No.: US 2025/0352691 A1
(43) Pub. Date: Nov. 20, 2025

(54) ANIONIC POLYMER-IMPREGNATED MATRIX FOR THE LOCALIZATION OF PHMB

(71) Applicant: MiMedx Group, Inc., Marietta, GA (US)

(72) Inventors: Thomas J. Koob, Bar Harbor, ME (US); Michelle Massee, Milton, GA (US); Jake Mullan, Phoenixville, PA (US)

(21) Appl. No.: 19/208,085

(22) Filed: May 14, 2025

Related U.S. Application Data
(60) Provisional application No. 63/647,317, filed on May 14, 2024.

(51) Int. Cl. A61L 15/44; A61L 15/22; C08L 5/08; C08L 79/00

(52) U.S. Cl. CPC A61L 15/44; A61L 15/225; C08L 5/08; C08L 79/00; A61L 2300/206; A61L 2300/404

(57) ABSTRACT

The invention provides for methods and compositions related to substrates used for wound care that incorporate bactericidal agents in a manner such that the agent can be anchored within the substrate, allowing for sustained protection from bacterial contamination and growth within said product throughout the duration of its usage.

### FIELD OF THE INVENTION

The invention relates to a wound care product impregnated with a bactericidal agent in a manner such that the agent can be anchored within the product, allowing for sustained protection of said product throughout the duration of its storage, application, and lifetime in situ once applied to the patient.

### BACKGROUND

Wound dressings often contribute to increased incidence and persistence of bacterial infection in recalcitrant, non-healing and chronic wounds as they may entrap bacteria and provide a bacterial milieu over the course of treatment. Chronic bacterial infection is one of the major causes of wound recalcitrance and non-healing. The covering itself may also become colonized and impair the healing process through recurring reinfection.

Polyhexamethylene biguanide (PHMB), also known as polyhexanide, is a potent bactericidal agent. PHMB-containing products are available on the market; however, upon hydration, the PHMB can rapidly leach from the product into the wound microenvironment, and/or leave the wound entirely, if not properly anchored within the product substrate. The bolus release may offer some initial benefit for fighting bacterial infection in the wound; however, the depleted product is vulnerable to colonization for the remainder of the treatment duration.

Accordingly, it would be desirable to develop wound dressings comprising stably-bound PHMB, so as to avoid colonization of the wound dressing with infectious agents and subsequent reinfection of the wound. It is with this and other considerations in mind that the presently described wound dressings and associated methods are provided.

### SUMMARY OF THE INVENTION

An embodiment of the invention is a method for anchoring a cationic, bactericidal agent into a wound care product through chemical interactions with an anionic polymer. An embodiment of the invention is ensuring protection from microbial colonization of the product throughout its indicated usage period by means of anchoring the bactericidal agent through reversible electrostatic interactions. The impermanent nature of these interactions allows for the agent to reversibly bind and unbind from the polymer in a concentration dependent manner. An influx of bacteria would prompt the release of PHMB, preventing colonization of the substrate. In certain embodiments the substrate is derived from human placental tissue, xenograft collagen tissue, or synthetic materials. In certain embodiments the wound care product is administered as a sheet/dressing, gauze, bandage, particulate, or gel.

### BRIEF DESCRIPTION OF THE FIGURES

Further features and benefits of the present invention will be apparent from a detailed description of preferred embodiments thereof taken in conjunction with the following drawings, wherein similar elements are referred to with similar reference numbers, and wherein:

FIG. 1 shows results from a neutralization assessment of Dressings 1 and 2 and Negative Control 3.

FIG. 2 shows results from the antimicrobial efficacy assessment of Dressings 1 and 2 against Staphylococcus aureus USA 300.

FIG. 3 shows results from the antimicrobial efficacy assessment of Dressings 1 and 2 against Pseudomonas aeruginosa ATCC 27312.

FIG. 4 shows results from the antimicrobial efficacy assessment of Dressings 1 and 2 against Candida albicans ATCC 10231.

FIG. 5 shows results from a neutralization assessment of Dressings 4 and 5 and Negative Control 6.

FIG. 6 shows results from the antimicrobial efficacy assessment of Dressings 4 and 5 against Staphylococcus aureus USA 300.

FIG. 7 shows results from the antimicrobial efficacy assessment of Dressings 4 and 5 against Escherichia coli ATCC 8739.

FIG. 8 shows results from the antimicrobial efficacy assessment of Dressings 4 and 5 against Candida albicans ATCC 10231.

### DETAILED DESCRIPTION OF THE INVENTION

It is to be understood that this invention is not limited to particular embodiments described, and as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the manufacture, practice or testing of the present invention, the preferred methods and materials are now described. All patents and publications mentioned herein are incorporated by reference to disclose and describe the methods and/or materials in connection with which the publications are cited.

Each embodiment disclosed herein is contemplated as being applicable to each of the other disclosed embodiments. All combinations and sub-combinations of the various elements described herein are within the scope of the embodiments.

It is understood that where a parameter range is provided, all integers and ranges within that range, and tenths and hundredths thereof, are also provided by the embodiments. For example, “5-10%” includes 5%, 6%, 7%, 8%, 9%, and 10%; 5.0%, 5.1%, 5.2% . . . 9.8%, 9.9%, and 10.0%; and 5.00%, 5.01%, 5.02% . . . 9.98%, 9.99%, and 10.00%, as well as, for example, 6-9%, 5.1%-9.9%, and 5.01%-9.99%. This also applies to ratios. For example, a recited ratio range of “1:100 to 200:1” includes ratios such as 1:50, 1:1, and 100:1, along with ranges such as 1:100 to 1:1, 1:50 to 50:1, and 1:1 to 200:1.

As used herein, “about” in the context of a numerical value or range means within ±1%, ±5%, or 10% of the numerical value or range recited or claimed.

The invention illustratively disclosed herein suitably may be practiced in the absence of any element which is not specifically disclosed herein.

It must be noted that as used herein and in the appended claims, the singular forms “a”, “an”, and “the” include plural referents unless the context clearly dictates otherwise.

### Definitions

As used herein the following terms have the following meanings.

“Comprising” or “comprises” is intended to mean that the compositions, for example media, and methods include the recited elements, but not excluding others. “Consisting essentially of” when used to define methods, shall mean excluding other elements of any essential significance to the combination for the stated purpose. “Consisting of” shall mean excluding additional substantial method steps. Embodiments defined by each of these transition terms are within the scope of this invention.

“Dehydrated” means that the tissue has had substantially all of its water removed, (i.e., greater than 85%, greater than 90%, greater than 95%, greater than 99%, or 100% of its water removed).

“Substantially uniform,” with respect to the intermediate layer thickness, means that the thickness is +20%, +15%, +10%, +5%, or +1% throughout the entirety of the graft.

“Optional” or “optionally” means that the subsequently described event or circumstance can or cannot occur, and that the description includes instances where the event or circumstance occurs and instances where it does not.

The term “subject” as used herein is any vertebrate organism including but not limited to mammalian subjects such as humans, farm animals, domesticated pets and the like. The term “patient” may be used interchangeably with “subject.”

The term “treat,” with respect to a wound, means to reduce the amount of time the wound would have taken to heal in the absence of any type of medical intervention.

The terms “substrate” or “substrate material” as used herein, refer to any material in which or on which a combination of PHMB and anionic polymer may be mixed, embedded, or deposited. In an embodiment, the substrate is porous. In an embodiment, the substrate is fibrous. The substrate material may be any insoluble material, naturally-occurring (including animal-derived) or synthetic, which can be impregnated with the PHMB and anionic polymer.

The term “antimicrobial,” as used herein, refers to any material which has the ability to kill or inhibit the growth of microorganisms. This includes, but is not limited to, bacteria (“antibacterial”), viruses (“antiviral”), and fungi (“antifungal”).

An embodiment of the invention is a sterile antimicrobial substrate, comprising:
a substrate material, and
a polyhexamethylene biguanide (PHMB)/anionic polymer complex,
wherein the PHMB/anionic polymer complex is disposed throughout at least a portion of the substrate material.

Another embodiment of the invention is a method of preparing an antimicrobial substrate comprising a polyhexamethylene biguanide (PHMB)/anionic polymer complex, comprising:
i) contacting a substrate material with an anionic polymer, thereby obtaining an anionic polymer-impregnated substrate; and
ii) contacting the anionic polymer-impregnated substrate with PHMB, in a manner permitting the PHMB to form a complex with the anionic polymer;
thereby obtaining an antimicrobial substrate comprising a PHMB/anionic polymer complex.

In an embodiment, said substrate material comprises a wound dressing, collagen, biocellulose, or a tissue graft. In an embodiment, the substrate material is in a form selected from the group consisting of a solid, an aqueous solution, or an aqueous suspension. In an embodiment, the substrate material is a solid. In an embodiment, the substrate material is an aqueous solution or an aqueous suspension.

In an embodiment, said wound dressing comprises synthetic material.

In an embodiment, said wound dressing is selected from the group consisting of gauze, a bandage, or a foam. In an embodiment, the wound dressing is affixed to one or more adhesive strips.

The collagen may be from any source. In an embodiment, the collagen is from an animal source. In an embodiment, said collagen is derived from mammalian tissue. In an embodiment, the mammalian tissue is human tissue. In an embodiment, the collagen is derived from a non-human (xenograft) animal source. In an embodiment, the collagen is derived from a non-human mammal source. In an embodiment, said collagen is synthetic collagen. In an embodiment, the collagen is selected from the group consisting of intact fibrillar collagen, purified collagen, denatured collagen, and gelatin.

In an embodiment, said tissue graft is derived from mammalian tissue. In an embodiment, the mammalian tissue is human tissue. In an embodiment, said tissue graft is a placental tissue graft.

In an embodiment, said wound dressing is in the form of a sheet, a particulate, a gauze, a bandage, a spray, an aqueous solution, an aqueous suspension, or a gel.

In an embodiment, said anionic polymer is a monoanionic polymer. In an alternative embodiment, said anionic polymer is a polyanionic polymer.

The polyanionic polymer may be any polymer comprising negatively charged side chains. In an embodiment, the side chains are sulfates, phosphates, carboxylates, or any combination thereof. In an embodiment, said polyanionic polymer is selected from the group consisting of chondroitin sulfate A, chondroitin sulfate C, dermatan sulfate, and heparan sulfate, fucoidan, carboxymethylcellulose and cellulose derivatives.

In an embodiment, said PHMB and said anionic polymer are present in a ratio of about 1:10 to about 10:1, by weight. In an embodiment, said ratio is at least, at most, or about 1:10, 1:5, 1:3, 1:2, 1:1.5, 1:1, 1.5:1, 2:1, 3:1, 5:1, or 10:1, or within a range defined by any two of these values.

In an embodiment, said substrate material and said PHMB/anionic polymer complex are present in a ratio of about 10:1 to about 10,000:1 by weight. In an embodiment, said ratio is at least, at most, or about 10:1, 20:1, 50:1, 100:1, 200:1, 500:1, 1,000:1, 2,000:1, 5,000:1, or 10,000:1, or within a range defined by any two of these values.

In an embodiment, the ratio of substrate material:PHMB:anionic polymer is about 10-10,000:0.1-10:0.1-10, by weight.

In an embodiment, the substrate material is in a form selected from the group consisting of a solid, an aqueous solution, or an aqueous suspension. In an embodiment, the substrate material is a solid. In an embodiment, the substrate material is an aqueous solution or an aqueous suspension.

In an embodiment, the antimicrobial substrate provides a sustained release of an effective amount of PHMB. In an embodiment, the antimicrobial substrate provides a sustained release of an effective amount of PHMB over a period of at least 1 day, at least 5 days, at least 7 days, at least 14 days, at least 21 days, at least 28 days, at least 30 days, at least 35 days, at least 40 days, at least 50 days, or at least 60 days. In an embodiment, the antimicrobial substrate provides a sustained release of an effective amount of PHMB over a period of about 1 day, about 5 days, about 7 days, about 14 days, about 21 days, about 28 days, about 30 days, about 35 days, about 40 days, about 50 days, or about 60 days.

Another embodiment of the invention is a method of preparing an antimicrobial substrate complex, comprising:
i) homogenizing a mixture of substrate material, PHMB, and anionic polymer, to obtain a homogenate, wherein the substrate material is selected from the group consisting of collagen and biocellulose; and
ii) dehydrating the homogenate,
thereby obtaining an antimicrobial substrate. In an embodiment, the antimicrobial substrate comprises a PHMB/anionic polymer complex.

In such embodiments, the order of combining the components is not critical. In one embodiment, an aqueous collagen slurry is combined with the PHMB and the anionic polymer, in any order. In an embodiment, the mixture is an aqueous mixture.

In an embodiment, the method further comprises crosslinking the homogenate. In an embodiment, said crosslinking is performed via chemical crosslinking and/or dehydrothermal (DHT) crosslinking. In an embodiment, the crosslinking is performed via DHT crosslinking.

In intact fibrillar type I collagen, two identical alpha chains, called the al-chain and the third chain called α2-chain, are twisted with each other into a right-handed triple helix structure. Every chain contains the repeating amino acids (Gly-X-Y), where the X and Y are frequently proline and hydroxyproline. Collagen molecules organize themselves to fibrils side by side with intrafibrillar crosslinking. These fibrils can form into fibers which can interweave into one-dimensional networks or two- and three-dimensional networks according to different functions. Collagen's exceptional tensile strength stems from its unique molecular structure, specifically the triple-helical arrangement of its polypeptide chains, and the formation of intermolecular crosslinks between collagen molecules.

Processing methods employed in the manufacture of collagen-based biomaterials are necessary to confer biocompatibility to a xenograft; however, in doing so, they often denature the collagen fibrils. The loss of mechanical properties correlates to the extent of damage incurred during processing.

Restoring tensile strength to a collagen matrix post-processing can be accomplished through secondary crosslinking techniques. The use of dehydrothermal crosslinking (DHT) is an attractive method because it leverages physical parameters to decrease the swelling or instability in the matrix. DHT does not introduce exogenous chemicals and therefore, unlike some chemical crosslinking methods, has no associated cytotoxicity. The process of DHT treatment entails exposing the collagen to a high temperature (up to 150° C.) under vacuum for a period of time to remove water and form crosslinks. The exact mechanism includes the formation of amide bonds between carboxyl and amine groups, as well as ester bonds between carboxyl and hydroxyl groups, via condensation reactions. In an embodiment, said anionic polymer is a monoanionic polymer. In an alternative embodiment, said anionic polymer is a polyanionic polymer.

In an embodiment, said polyanionic polymer is selected from the group consisting of chondroitin sulfate A, chondroitin sulfate C, dermatan sulfate, and heparan sulfate, fucoidan, carboxymethylcellulose and cellulose derivatives.

In an embodiment, said PHMB and said anionic polymer are present in a ratio of about 1:10 to about 10:1, by weight. In an embodiment, said ratio is at least, at most, or about 1:10, 1:5, 1:3, 1:2, 1:1.5, 1:1, 1.5:1, 2:1, 3:1, 5:1, or 10:1, or within a range defined by any two of these values.

In an embodiment, the ratio of substrate material:PHMB:anionic polymer is about 10-10,000:0.1-10:0.1-10, by weight.

An embodiment is a method of treating a wound in a subject in need thereof, said method comprising applying an antimicrobial substrate as described above to the wound. In an embodiment, the antimicrobial substrate provides a sustained release of an effective amount of PHMB over a period of at least 1 day, at least 5 days, at least 7 days, at least 14 days, at least 21 days, at least 28 days, at least 30 days, at least 35 days, at least 40 days, at least 50 days, or at least 60 days. In an embodiment, the antimicrobial substrate provides a sustained release of an effective amount of PHMB over a period of about 1 day, about 5 days, about 7 days, about 14 days, about 21 days, about 28 days, about 30 days, about 35 days, about 40 days, about 50 days, or about 60 days.

### DISCUSSION AND EXPERIMENTAL

As discussed above, in one embodiment of preparing the antimicrobial substrates of the invention, a substrate (for example, xenograft wound dressings, collagen dressings, collagen, biocellulose, gauze or any synthetic material used in wound management) is saturated with a concentration of an anionic polymer (natural anionic polymers like chondroitin sulfates A & C, dermatan sulfate, heparan sulfate, fucoidan, carboxymethylcellulose and cellulose derivatives; modified natural anionic polymers, organic and non-organic synthetic anionic polymers). The subsequent addition of a PHMB solution results in the electrostatic interaction with the polymer to create a precipitate within the substrate. The solid precipitate of PHMB and polymer prevents the solid from rapidly eluting from the matrix. The amount and size of the anionic polymer, coupled with amount of PHMB can be designed to provide optimal bactericidal activity over time. The resultant substrate may be crosslinked to tune the elution properties of the PHMB.

In alternative embodiments of preparing antimicrobial substrates of the invention, the substrate material, the PHMB, and the anionic polymer are combined in a slurry or solution.

Studies were conducted to determine the antimicrobial efficacy of dressings against a panel of microbial species. In the first study, two PHMB-containing collagen dressings (Dressing 1 and Dressing 2), along with a representative negative control without PHMB (Negative Control 3), were evaluated against one Gram-positive bacterium (Staphylococcus aureus USA300), one Gram-negative bacterium (Pseudomonas aeruginosa ATCC 27312), and one yeast (Candida albicans ATCC 10231). For the portions of the dressing used to assess the anti-microbial properties, Dressing 1 and Dressing 2 comprised 0.89% w/w of PHMB and 1.8% w/w of PHMB respectively in relation to the entire dressing.

### Materials and Methods

1 inch×0.5 inch sterilized dressings were used for the study. All measurements were tested in quadruplicate unless otherwise noted. The consumables and reagents used in this project were obtained from various suppliers and were used in sterile form, where applicable. All media and agar used in the study were prepared according to manufacturer recommendations and following internal protocols. A proprietary formula was used to prepare the simulated wound fluid (SWF), which was then utilized to prepare the microbial inoculum.

### Example 1: Absorption Test

To establish inoculation volumes, the absorption capacity of each dressing was assessed. Briefly, SWF was added to 1 inch×1 inch dressings (n=3) until dressings were completely saturated. After saturation, the excess SWF was allowed to drip off the dressings. All dressings were weighed before and after the addition of SWF. The absorption capacity of each dressing was calculated as follows:

(Mass wet dressing (g)−mass dry dressing (g))/mass dry dressing (g)

For this study, the total inoculation volume to add to each sample was calculated as 80% of the absorption capacity to deliver approximately 106 CFUs of microorganism. Table 1, below, shows a summary of the absorption assessment.

TABLE 1
Summary of absorption assessment.

| | Absorption | Inoculation Volume |
|---|---|---|
| Test Article | Capacity | (μL/sample) (80% of |
| (1 inch × 0.5 inch) | (μL/sample) | Absorption Capacity) |
| Dressing 1 | 317.5 | 254 |
| Dressing 2 | 407.5 | 326 |
| Negative Control 3 | 385 | 308 |

### Example 2: Neutralization Assessment

Before evaluating the antimicrobial activity of the test articles, a neutralization assessment was performed on Dressing 1, Dressing 2, and the negative control (Negative Control 3) and for all three microbial species (Staphylococcus aureus USA300, Pseudomonas aeruginosa ATCC 27312, and Candida albicans ATCC 10231). Briefly, the dressings were cut aseptically to approximately 1×0.5 inch in size. Dressings were then placed in sterile 6-well plates. According to the table 3, 254 μL, 326 μL, and 308 μL of SWF was added to Dressing 1, Dressing 2, and the negative control dressing samples, respectively, individually to mimic inoculation. The samples were then transferred into 50 mL centrifuge tubes filled with 20 mL of Dey Engley (D/E) recovery broth containing approximately 10⁴ CFUs of test microorganism. After a 30-minute incubation at room temperature, the samples were sonicated for 20 minutes and 100 μL of each sample was spread plated. The spread plates were incubated at 37° C. prior to enumeration. The neutralization efficacy was evaluated in triplicate for each species. If the recovery concentration of microorganisms is within 70-130% of the negative control, the neutralization is considered successful.

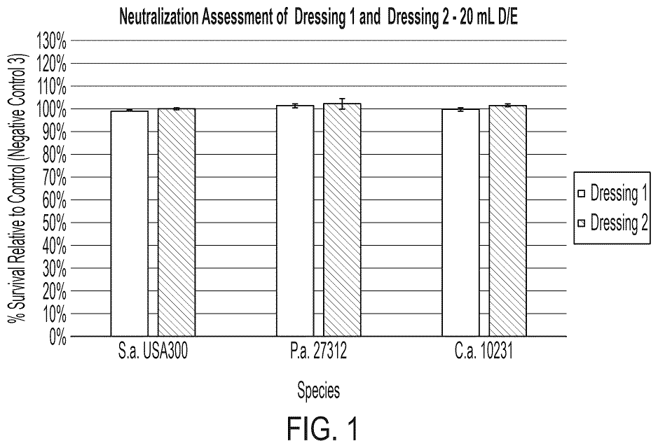

A summary of the neutralization data is shown in FIG. 1 as percent survival relative to the inoculum concentration. All test species met the acceptance criteria for successful neutralization. The shaded zone on the graph shows the acceptable range for successful neutralization (CFU 70-130% of the inoculum). The CFU recovered from the test dressing fell between 70% to 130% of the inoculum.

### Experiment 3: Antimicrobial Testing (Modified AATCC 100 Test Method)

Dressings were cut aseptically to approximately 1 inch×0.5 inch in size. The dressings were placed in sterile 6-well plates. Subsequently, Dressing 1, Dressing 2, and the negative control dressing were inoculated with 254 μL, 326 μL, and 308 μL of simulated wound fluid (SWF), respectively, containing approximately 106 CFUs of the test microorganism. For each assessment, the inoculum was enumerated. Immediately following inoculation, t=0 samples were recovered by transferring the samples to individual 50 mL centrifuge tubes containing 20 mL of D/E broth. The samples underwent sonication for 20 minutes, followed by serial dilution and plating for enumeration. The remaining dressings were incubated for an additional 24 hours at 37° C., then transferred individually to 50 mL centrifuge tubes containing 20 mL of D/E broth for sonication, recovery, and plating as described above as well as 200 μL spread plates. Each dressing was evaluated in quadruplicate, and the experiment was performed once per species. The plates for enumeration were incubated at 37° C. The limit of detection for this study was 50 CFU/mL.

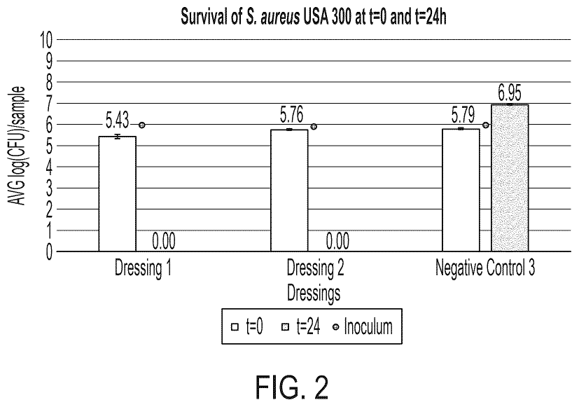

The data from the antimicrobial efficacy assessments are summarized below and shown in FIGS. 2-4 as survival in log 10 (CFU)/dressing. To determine microbial reduction after 24 hours, the data were compared with the negative control dressing value at t=24 hr for the corresponding species. Each error bar in FIGS. 2-4 represents the standard deviation across 4 sample replicates.

FIG. 2 depicts the antimicrobial efficacy of Dressings 1 and 2 against Staphylococcus aureus USA 300. Analysis of microbial load reduction data reveals that, following a 24-hour contact period, both Dressings 1 and 2 exhibited antimicrobial activity beyond the limit of detection and achieved microbial reductions of ≥4 logs. No surviving Staphylococcus aureus USA 300 was recovered from Dressings 1 and 2 within the limit of detection. In contrast, the microbial load of the negative control dressing (no active agent present), increased from 5.79 (t=0) to 6.95 log (t=24 h). Results are also shown in Table 2, below.

TABLE 2
Results from the antimicrobial efficacy assessment of Dressings
1 and 2 against Staphylococcus aureus USA 300.
T = 24 h Survival

| | Log | | Log |
|---|---|---|---|
| Dressing | (CFU)/Sample | SD | Reduction |
| Negative control 3 | 6.95 | 0.02 | N/A |
| Dressing 1 (t = 24 h) | 0.00 | 0.00 | 6.95 |
| Dressing 2 (t = 24 h) | 0.00 | 0.00 | 6.95 |

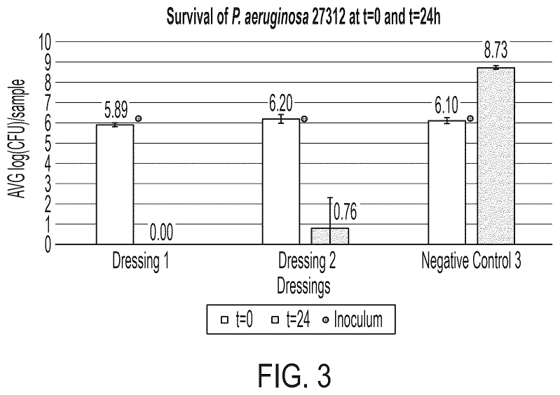

FIG. 3 depicts the antimicrobial efficacy of Dressings 1 and 2 against Pseudomonas aeruginosa ATCC 27312. Analysis of microbial load reduction data reveals that, following a 24-hour contact period, both Dressings 1 and 2 exhibited antimicrobial activity beyond the limit of detection and achieved microbial reductions of >4 logs. No surviving Pseudomonas aeruginosa ATCC 27312 was recovered from Dressings 1 and 2 within the limit of detection. In contrast, the microbial load of the negative control dressing (no active agent present), increased from 6.10 (t=0) to 8.73 log (t=24 h). Results are also shown in Table 3, below.

TABLE 3
Results from the antimicrobial efficacy assessment of Dressings
1 and 2 dressings against Pseudomonas Aeruginosa ATCC 27312
T = 24 h Survival

| | Log | | Log |
|---|---|---|---|
| Dressing | (CFU)/Sample | SD | Reduction |
| Negative control 3 (t = 24 h) | 8.73 | 0.09 | N/A |
| Dressing 1 (t = 24 h) | 0.00 | 0.00 | 8.73 |
| Dressing 2 (t = 24 h) | 0.76 | 1.52 | 7.97 |

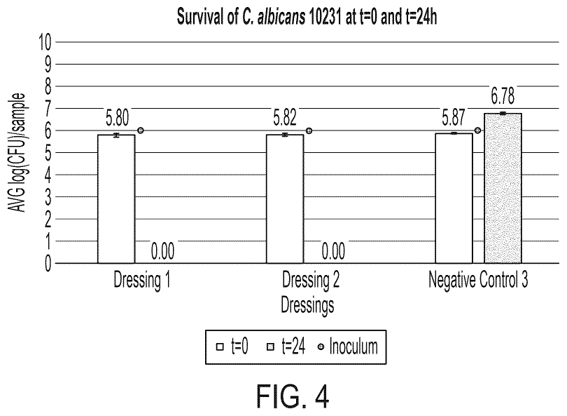

FIG. 4 depicts the antimicrobial efficacy of Dressings 1 and 2 against Candida albicans ATCC 10231. Analysis of microbial load reduction data reveals that, following a 24-hour contact period, both Dressings 1 and 2 exhibited antimicrobial activity beyond the limit of detection and achieved microbial reductions of ≥4 logs. No surviving Candida albicans ATCC 10231 was recovered from Dressings 1 and 2 within the limit of detection. In contrast, the microbial load of the negative control dressing (no active agent present), increased from 5.87 (t=0) to 6.78 log (t=24 h). Results are also shown in Table 4, below.

TABLE 4
Results from the antimicrobial efficacy assessment of Dressings
1 and 2 dressings against Candida albicans ATCC 10231
T = 24 h Survival

| | Log | | Log |
|---|---|---|---|
| Dressing | (CFU)/Sample | SD | Reduction |
| Negative control 3 (t = 24 h) | 6.78 | 0.04 | N/A |
| Dressing 1 (t = 24 h) | 0.00 | 0.00 | 6.78 |
| Dressing 2 (t = 24 h) | 0.00 | 0.00 | 6.78 |

### Summary

Two PHMB-containing dressings, along with a representative negative control, were evaluated against one Gram-positive bacterium, one Gram-negative bacterium, and one yeast. The test samples were inoculated with approximately 106 CFUs of bacteria/yeast and incubated for 24 hours. The surviving organisms were recovered and enumerated immediately after inoculation at t=0 and after 24 hours. Overall, both antimicrobial-containing dressings demonstrated excellent antimicrobial efficacy against all three species, achieving a microbial reduction of ≥4 logs.

A second study was conducted to determine the antimicrobial efficacy of dressings against a panel of microbial species, both with and without preconditioning in SWF. Two PHMB-containing dressings (Dressing 4 and Dressing 5), along with a representative negative control without PHMB (Negative Control 6), were evaluated against one Gram-positive bacterium (Staphylococcus aureus USA300), one Gram-negative bacterium (Escherichia coli ATCC 8739), and one yeast (Candida albicans ATCC 10231). For the portions of the dressing used to assess the anti-microbial properties, Dressing 4 and Dressing 5 comprised 0.89% w/w of PHMB and 1.8% w/w of PHMB respectively in relation to the entire dressing.

### Materials and Methods

1 inch×0.5 inch sterilized dressings were used for the study. All measurements were tested in quadruplicate unless otherwise noted. The consumables and reagents used in this project were obtained from various suppliers and were used in sterile form, where applicable. All media and agar used in the study were prepared according to manufacturer recommendations and following internal protocols. A proprietary formula was used to prepare the simulated wound fluid (SWF), which was then utilized to prepare the microbial inoculum.

### Experiment 4: Absorption Test

To establish inoculation volumes, the absorption capacities of each dressing was assessed in a manner similar to that described in Example 1. For this study, the total inoculation volume to add to each sample was calculated as 20% of 80% of the absorption capacity (127 μL) to deliver approximately 106 CFUs of microorganism.

In addition to absorption capacity testing, it was also evaluated how to make room for adding microbial inoculum after 3 or 6 days of preconditioning. Since the preconditioned samples will be fully saturated after these periods, there will be no room for adding 127 μL of microbial inoculum. It was determined that applying one 3×3 cm and one 3×1.5 cm sterile filter paper can absorb enough SWF from one saturated dressing to allow for the addition of the inoculum.

Table 5, below, shows a summary of the absorption assessment.

TABLE 5
Summary of absorption assessment.

| | Absorption | Inoculation Volume |
|---|---|---|
| Test Article | Capacity | (μL/sample) (20% to 80% |
| (1 inch × 1 inch) | (μL/sample) | of Absorption Capacity) |
| Dressing 4 | 817 | 126.8 |
| Dressing 5 | 793 | 126.8 |
| Negative Control 6 | 768 | 126.8 |

### Experiment 5: Neutralization Assessment

Before evaluating the antimicrobial activity of the test articles, a neutralization assessment was performed on Dressings 4 and 5 for all three microbial species (Staphylococcus aureus USA300, Escherichia coli ATCC 8739, and Candida albicans ATCC 10231), along with the negative control (Negative Control 6). Briefly, 1×1 inch dressings were aseptically placed in sterile 6-well plate. Samples were hydrated by adding 634 μL (80% of the absorption capacity) of SWF. The samples were then transferred into 50 mL centrifuge tubes filled with 20 mL of Dey Engley (D/E) recovery broth containing approximately 10³ CFUs of test microorganism. After a 30-minute incubation at room temperature, the samples were sonicated for 20 minutes and 100 μL of each sample was spread plated. The spread plates were incubated at 37° C. prior to enumeration. The neutralization efficacy was evaluated in triplicate for each species. If the recovery concentration of microorganisms is within 70-130% of the negative control, the neutralization is considered successful.

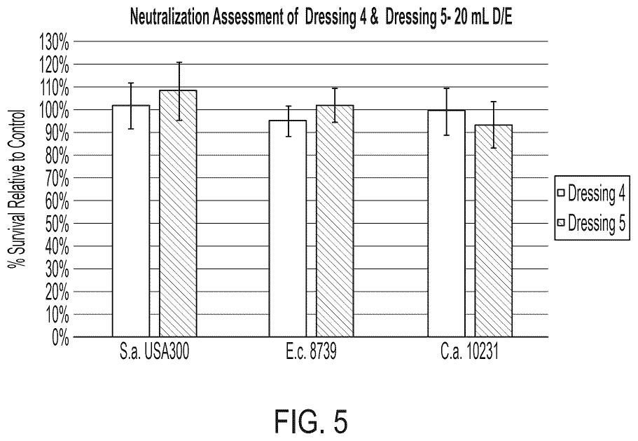

A summary of the neutralization data is shown in FIG. 5 as percent survival relative to Negative Control 6. All test species met the acceptance criteria for successful neutralization. The CFU recovered from the test dressing fell between 70% to 130% of the inoculum.

### Experiment 6: Antimicrobial Testing (Modified AATCC 100 Test Method)

The data from the antimicrobial efficacy assessments are summarized in the following sections and shown as survival in log 10 (CFU)/dressing. To determine microbial reduction after 24 hours, the data were compared with the T=0 microbial survival for the corresponding dressings. Each error bar represents the standard deviation across 4 sample replicates.

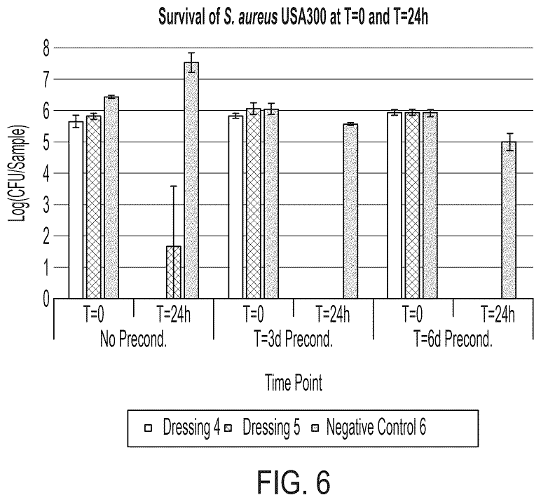

FIG. 6 depicts the antimicrobial efficacy of Dressings 4 and 5 against Staphylococcus aureus USA 300. For non-preconditioned samples, after a 24-hour contact period, Dressings 4 and 5 dressings showed a 5.64 and 4.13 log reduction, respectively. No surviving Staphylococcus aureus USA 300 was recovered from Dressing 4 after the 24-hour contact period. However, 1.66 log of Staphylococcus aureus USA 300 was recovered from Dressing 5. Out of four replicates, two showed full kill, while the other two showed 3.26 and 3.38 log survival. In contrast, the microbial load of the negative control dressing (with no active agent present), Negative Control 6, increased from 6.40 (at T=0) to 7.51 log (at T=24 h). Results are also shown in Table 6, below.

TABLE 6
Results from the antimicrobial efficacy assessment of Dressings
4 and 5 dressings against Staphylococcus aureus USA 300

| | | T = 0 Survival | T = 24 h Survival | |
|---|---|---|---|---|
| Precondi- | | Log(CFU)/ | Log(CFU)/ | Log |
| tioning | Dressing | Sample ± SD | Sample ± SD | Reduction |
| None | Dressing 4 | 5.64 ± 0.20 | 0.00 ± 0.00 | 5.64 |
| | Dressing 5 | 5.79 ± 0.10 | 1.66 ± 0.00 | 4.13 |
| 3 days | Dressing 4 | 5.81 ± 0.08 | 0.00 ± 0.00 | 5.81 |
| | Dressing 5 | 6.04 ± 0.18 | 0.00 ± 0.00 | 6.04 |
| 6 days | Dressing 4 | 5.92 ± 0.09 | 0.00 ± 0.00 | 5.92 |
| | Dressing 5 | 5.93 ± 0.09 | 0.00 ± 0.00 | 5.93 |

Samples preconditioned for 3 days also showed excellent efficacy against Staphylococcus aureus USA 300. After a 24-hour contact period, Dressings 4 and 5 showed a 5.81 and 6.04 log reduction, respectively. No surviving Staphylococcus aureus USA 300 was recovered from Dressings 4 and 5 after the 24-hour contact period. In contrast, the microbial load of the negative control dressing (with no active agent present), Negative Control 6, was static around 6 logs at T=0 and T=24 h.

Similar to the non-preconditioned and 3-day preconditioned samples, samples preconditioned for 6 days also demonstrated excellent efficacy against Staphylococcus aureus USA 300. After a 24-hour contact period, Dressings 4 and 5 showed a 5.92 and 5.93 log reduction, respectively. No surviving Staphylococcus aureus USA 300 was recovered from Dressings 4 and 5 after the 24-hour contact period. In contrast, the microbial load of the negative control dressing (with no active agent present), Negative Control 6, slightly decreased from 5.91 log (T=0) to 4.99 log (T=24 h).

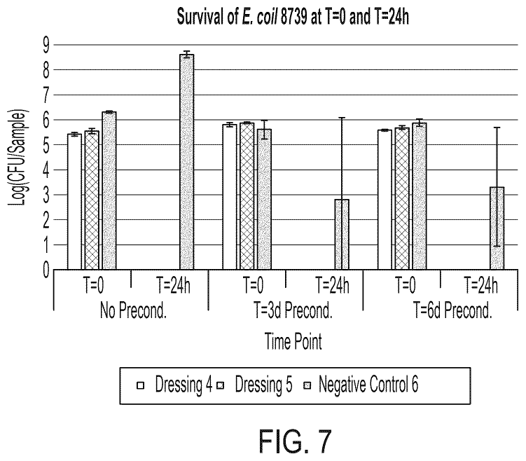

FIG. 7 depicts the antimicrobial efficacy of Dressings 4 and 5 against Escherichia coli ATCC 8739. For non-preconditioned samples, after a 24-hour contact period, Dressings 4 and 5 showed a 5.43 and 5.55 log reduction, respectively. No surviving Escherichia coli ATCC 8739 was recovered from Dressings 4 and 5 after the 24-hour contact period. In contrast, the microbial load of the negative control dressing (with no active agent present), Negative Control 6, increased from 6.31 (at T=0) to 8.63 log (at T=24 h). Results are also shown in Table 7, below.

TABLE 7
Results from the antimicrobial efficacy assessment of Dressings
4 and 5 dressings against Escherichia coli ATCC 8739

| | | T = 0 Survival | T = 24 h Survival | |
|---|---|---|---|---|
| Precondi- | | Log(CFU)/ | Log(CFU)/ | Log |
| tioning | Dressing | Sample ± SD | Sample ± SD | Reduction |
| None | Dressing 4 | 5.43 ± 0.06 | 0.00 ± 0.00 | 5.43 |
| | Dressing 5 | 5.55 ± 0.10 | 0.00 ± 0.00 | 5.55 |
| 3 days | Dressing 4 | 5.81 ± 0.09 | 0.00 ± 0.00 | 5.81 |
| | Dressing 5 | 5.88 ± 0.03 | 0.00 ± 0.00 | 5.88 |
| 6 days | Dressing 4 | 5.60 ± 0.02 | 0.00 ± 0.00 | 5.60 |
| | Dressing 5 | 5.71 ± 0.07 | 0.00 ± 0.00 | 5.71 |

Samples preconditioned for 3 days also showed efficacy against Escherichia coli ATCC 8739. After a 24-hour contact period, Dressings 4 and 5 showed a 5.81 and 5.88 log reduction, respectively. No surviving Escherichia coli ATCC 8739 was recovered from Dressings 4 and 5 after the 24-hour contact period. However, for the negative control dressing (with no active agent present), Negative Control 6, we observed variability in microbial recovery at T=24 h. Out of four replicates, two showed full kill, while the other two showed 5.8 and 5.54 log survival. Even though T=24 h recovery demonstrated variability, T=0 recovery was consistent with 5.61 log survival.

Samples preconditioned for 6 days also showed efficacy against Escherichia coli ATCC 8739. After a 24-hour contact period, Dressings 4 and 5 showed a 5.60 and 5.71 log reduction, respectively. No surviving Escherichia coli ATCC 8739 was recovered from Dressings 4 and 5 after the 24-hour contact period. However, for negative control dressing (with no active agent present), Negative Control 6, variability was observed in microbial recovery at T=24 h. Out of four replicates, one showed full kill, while the other three showed 5.62, 3.85, and 3.79 log survival. Even though T=24 h recovery demonstrated variability, T=0 recovery was consistent with 5.89 log survival.

Compared to the other two species tested in this study, Escherichia coli ATCC 8739 demonstrated high variability in T=24 h recovery for preconditioned Negative Control 6 samples. Interestingly, for the same preconditioned samples, T=0 recovery for Negative Control 6 dressings was consistent, with approximately 6 logs recovered. Although T=24 h recovery for the preconditioned samples showed variability, no such variability was observed for T=24 h non-preconditioned Negative Control 6 samples. The microbial load for non-preconditioned Negative Control 6 samples increased from 6.31 log (T=0) to 8.63 log (T=24 h). The variability and reduced viability of Escherichia coli ATCC 8739 on preconditioned Negative Control 6 samples at T=24 h require further investigation. This could be due to the interaction between Negative Control 6 and SWF during preconditioning, making Escherichia coli ATCC 8739 survival difficult. Alternatively, more nutrient-rich SWF can be evaluated for Escherichia coli ATCC 8739 survival on preconditioned samples.

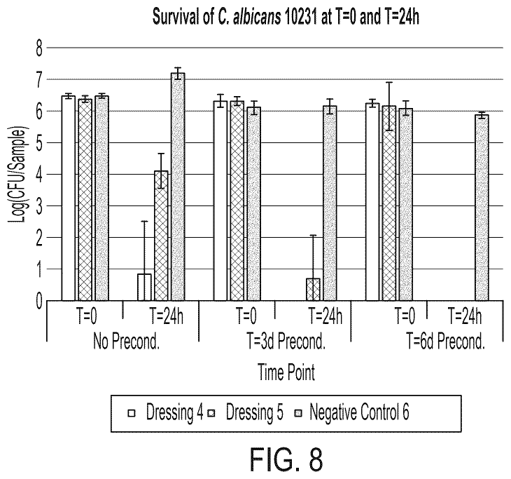

FIG. 8 depicts the antimicrobial efficacy of Dressings 4 and 5 against Candida albicans ATCC 10231. For non-preconditioned samples, after a 24-hour contact period, Dressing 4 showed a 5.63 log reduction, with three replicates showing no survivors and one replicate with 3.36 log surviving Candida albicans ATCC 10231. On the other hand, Dressing 5 showed a 2.27 log reduction with an average of 4.11 log survival across four replicates. In contrast, the microbial load of the negative control dressing (with no active agent present), Negative Control 6, increased from 6.49 (at T=0) to 7.19 log (at T=24 h). Results are also shown in Table 8, below.

TABLE 8
Results from the antimicrobial efficacy assessment of Dressings
4 and 5 dressings against Candida albicans ATCC 10231

| | | T = 0 Survival | T = 24 h Survival | |
|---|---|---|---|---|
| Precondi- | | Log(CFU)/ | Log(CFU)/ | Log |
| tioning | Dressing | Sample ± SD | Sample ± SD | Reduction |
| None | Dressing 4 | 6.47 ± 0.08 | 0.84 ± 1.68 | 5.63 |
| | Dressing 5 | 6.39 ± 0.11 | 4.11 ± 0.55 | 2.27 |
| 3 days | Dressing 4 | 6.32 ± 0.21 | 0.00 ± 0.00 | 6.32 |
| | Dressing 5 | 6.32 ± 0.14 | 0.69 ± 1.39 | 5.62 |
| 6 days | Dressing 4 | 6.24 ± 0.12 | 0.00 ± 0.00 | 6.24 |
| | Dressing 5 | 6.15 ± 0.75 | 0.00 ± 0.00 | 6.15 |

Samples preconditioned for 3 days also showed excellent efficacy against Candida albicans ATCC 10231. After a 24-hour contact period, Dressings 4 and 5 showed a 6.32 and 5.62 log reduction, respectively. No surviving Candida albicans ATCC 10231 was recovered from Dressing 4, while one of the four Dressing 5 replicates showed 2.77 log survival after the 24-hour contact period. In contrast, the microbial load of the negative control dressing (with no active agent present), Negative Control 6, remained static around 6 logs at T=0 and T=24 h.

Similar to the non-preconditioned and 3-day preconditioned samples, samples preconditioned for 6 days also demonstrated excellent efficacy against Candida albicans ATCC 10231. After a 24-hour contact period, Dressings 4 and 5 showed a 6.24 and 6.15 log reduction, respectively. No surviving Candida albicans ATCC 10231 was recovered from Dressings 4 and 5 after the 24-hour contact period. In contrast, the microbial load of the negative control dressing (with no active agent present), Negative Control 6, remained static around 6 logs at T=0 and T=24 h.

### Example 7: Extended Release

The ability of compositions comprising collagen, chondroitin sulfate, and PHMB (at high and low concentrations) to release PHMB in an extended release manner, relative to dressings comprising only collagen and PHMB, but not CS.

Collagen compositions were prepared by mixing a fixed amount of 0.05 wt. % PHMB or 0.10 wt. % PHMB solution with the collagen. For the collagen compositions that were also to comprise CS, the collagen was then coated with an 0.1% w/v solution of CS.

The method involved soaking the dressings in sterile water for the defined time points and then collecting the elution media following the duration of the elution period. All processing was performed at ambient temperature.

The results may be seen below in Table 9. Results marked as “*” were somewhere under the limit of quantification (LOQ) but above the limit of detection (LOD) (10-49 μg/mL), and results marked as “**” were under the LOD (0-9 μg/mL).

TABLE 9
Results of extended release testing

| | 1 Hour | 1 Day | 4 Day | 7 Day |
|---|---|---|---|---|
| Sample | μg/mL) | (μg/mL) | (μg/mL) | (μg/mL) |
| 0.05% PHMB ONLY | * | 67 | * | 59 |
| | * | 70 | 69 | 78 |
| 0.05% PHMB + CS | ** | * | * | * |
| | * | * | * | * |
| 0.1% PHMB ONLY | 81 | 168 | 177 | 181 |
| | 101 | 173 | 170 | 167 |
| 0.1% PHMB + CS | * | 77 | 80 | 82 |
| | * | 75 | 84 | 83 |

It can be seen that the dressings comprising CS eluted less PHMB than did the dressings lacking CS at the tested time points, indicating that the presence of CS allowed the collagen dressings to more effectively retain PHMB, and subsequently release it more gradually.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation. One skilled in the art will recognize many methods and materials similar or equivalent to those described herein, which could be used in the practicing the subject matter described herein. The present disclosure is in no way limited to just the methods and materials described.

1. A sterile antimicrobial substrate, comprising:
i) a substrate material, and
ii) a polyhexamethylene biguanide (PHMB)/anionic polymer complex,
wherein the PHMB/anionic polymer complex is disposed throughout at least a portion of the substrate material.

2. The antimicrobial substrate of claim 1, wherein said substrate material comprises a wound dressing, collagen, biocellulose, or a tissue graft.

3-5. (canceled)

6. The antimicrobial substrate of claim 1, wherein the tissue graft is derived from human tissue.

7. The antimicrobial substrate of claim 2, wherein said tissue graft is a placental tissue graft.

8. The antimicrobial substrate of claim 2, wherein said wound dressing is in the form of a sheet, a particulate, a gauze, a bandage, a spray, an aqueous solution, an aqueous suspension, a foam, or a gel.

9. (canceled)

10. The antimicrobial substrate of claim 1, wherein said anionic polymer is a polyanionic polymer selected from the group consisting of chondroitin sulfate A, chondroitin sulfate C, dermatan sulfate, and heparan sulfate, fucoidan, carboxymethylcellulose, and cellulose derivatives.

11. (canceled)

12. The antimicrobial substrate of claim 1, wherein PHMB and said anionic polymer are present in a ratio of about 1:10 to about 10:1, by weight.

13. (canceled)

14. (canceled)

15. The antimicrobial substrate material of claim 1, wherein the substrate material is in a form selected from the group consisting of a solid, an aqueous solution, and an aqueous suspension.

16. The antimicrobial substrate of claim 1, wherein the antimicrobial substrate provides a sustained release of an effective amount of PHMB.

17. A method of preparing an antimicrobial substrate comprising a polyhexamethylene biguanide (PHMB)/anionic polymer complex, comprising:
(A)
i) contacting a substrate material with an anionic polymer, thereby obtaining an anionic polymer-impregnated substrate; and
ii) contacting the anionic polymer-impregnated substrate with PHMB, in a manner permitting the PHMB to form a complex with the anionic polymer; or
(B)
i) homogenizing a mixture of substrate material, PHMB, and anionic polymer, to obtain a homogenate, wherein the substrate material is selected from the group consisting of collagen and biocellulose; and
ii) dehydrating the homogenate,
thereby obtaining an antimicrobial substrate comprising a PHMB/anionic polymer complex.

18. The method of claim 17, wherein said substrate material comprises a wound dressing, collagen, biocellulose, or a tissue graft.

19.-21. (canceled)

22. The method of claim 17, wherein the tissue graft is derived from human tissue.

23. The method of claim 18, wherein said tissue graft is a placental tissue graft.

24. The method of claim 18, wherein said wound dressing is in the form of a sheet, a particulate, a gauze, a bandage, a foam, or a gel.

25. (canceled)

26. The method of claim 17, wherein said anionic polymer is a polyanionic polymer selected from the group consisting of chondroitin sulfate A, chondroitin sulfate C, dermatan sulfate, and heparan sulfate, fucoidan, carboxymethylcellulose and cellulose derivatives.

27. (canceled)

28. The method of claim 17, wherein PHMB and said anionic polymer are present in a ratio of about 1:10 to about 10:1, by weight.

29. (canceled)

30. (canceled)

31. The method of claim 17, wherein the substrate material is in a form selected from the group consisting of a solid, an aqueous solution, and an aqueous suspension.

32. (canceled)

33. The method of claim 17, further comprising crosslinking the homogenate.

34.-39. (canceled)

40. A method of treating a wound in a subject in need thereof, said method comprising applying the antimicrobial substrate of claim 1 to the wound.

41. The method of claim 40, wherein the antimicrobial substrate releases an effective amount of PHMB for at least 1 day.

42. (canceled)
